SEQUENCE LISTING (5 sequences)

SEQ ID NO: 1 - protein, 5 aa, Gallus gallus
ILPEY

SEQ ID NO: 2 - protein, 5 aa, Gallus gallus
ILELP

SEQ ID NO: 3 - protein, 8 aa, Gallus gallus
LYRGGLEP

SEQ ID NO: 4 - protein, 4 aa, Gallus gallus
LPEY

SEQ ID NO: 5 - protein, 4 aa, Gallus gallus
ILPE

(19) United States
(12) Patent Application Publication
(10) Pub. No.: US 2025/0026786 A1
(43) Pub. Date: Jan. 23, 2025

(54) PEPTIDE-CONTAINING COMPOSITION

(71) Applicants: Kyoto University, Kyoto-shi, Kyoto (JP); KAZUSA DNA RESEARCH INSTITUTE, Kisarazu-shi, Chiba (JP)

(72) Inventors: Kousaku OHINATA, Kyoto-shi, Kyoto (JP); Takanobu NAKAJIMA, Kyoto-shi, Kyoto (JP); Maiko SHOBAKO, Kyoto-shi, Kyoto (JP); Kentaro KANEKO, Kyoto-shi, Kyoto (JP); Masaru SATO, Kisarazu-shi, Chiba (JP); Atsushi KURABAYASHI, Kisarazu-shi, Chiba (JP)

(21) Appl. No.: 18/714,413

(22) PCT Filed: Nov. 25, 2022
(86) PCT No.: PCT/JP2022/043509

§ 371 Date: May 29, 2024

(30) Foreign Application Priority Data
Nov. 30, 2021 (JP) 2021-194672

(51) Int. Cl. C07K 7/06; A61K 9/00; A61K 38/00; A61P 25/28; C07K 5/062; C07K 5/083; C07K 5/103

(52) U.S. Cl. CPC C07K 7/06; A61K 9/0053; A61P 25/28; C07K 5/06017; C07K 5/0804; C07K 5/1005; A61K 38/00

(57) ABSTRACT

Provided is a cognitive function-improving composition. Specifically, provided is a cognitive function-improving composition comprising at least one selected from the group consisting of a thermolysin digest of ovalbumin, a peptide comprising amino acid sequence LP, and a peptide comprising amino acid sequence LE.

### TECHNICAL FIELD

The present disclosure relates to a peptide-containing composition and the like.

### BACKGROUND ART

Japan has the highest aging rate in the world (28.7%, surveyed by the Ministry of Internal Affairs and Communications in September 2020), and it is strongly hoped that a society of health and longevity will be achieved. On the other hand, the number of dementia patients is expected to exceed 7 million by 2025, and dementia is becoming a common disease. In dementia, once dysfunction due to neurodegeneration occurs, it is not easy to improve the condition, so it is considered important to prevent neuronal dysfunction before it occurs. For this reason, the search for materials that improve cognitive function is underway (PTL 1 and NPL 1).

### CITATION LIST

### Patent Literature

PTL 1: WO2019/168149

### Non-Patent Literature

NPL 1: Nagai et al., FASEB J. December 2019; 33(12): 14095-14102

### SUMMARY OF INVENTION

### Technical Problem

An object of the present disclosure is to provide a cognitive function-improving composition.

### Solution to Problem

The present inventors have previously demonstrated that cognitive function declines in mice when they are fed a high-fat diet for one week (NPL 1). The present inventors found that by administering a thermolysin digest of ovalbumin, which is an egg white protein, to mice in the early stages of cognitive decline, cognitive function improved.

The present disclosure includes, for example, the subject matter described in the following items.
Item 1.

A cognitive function-improving composition comprising a thermolysin digest of ovalbumin.
Item 2.

The cognitive function-improving composition according to Item 1, wherein the thermolysin digest of ovalbumin comprises a peptide comprising amino acid sequence LP and/or a peptide comprising amino acid sequence LE.
Item 3.

The composition according to Item 2, wherein the number of amino acid residues that constitute the peptide comprising amino acid sequence LP and/or the peptide comprising amino acid sequence LE is 2 to 15.
Item 4.

The cognitive function-improving composition according to Item 1, wherein the thermolysin digest of ovalbumin comprises at least one peptide selected from the group consisting of:
a peptide consisting of amino acid sequence ILPEY (SEQ ID NO: 1),
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LPEY (SEQ ID NO: 4),
a peptide consisting of amino acid sequence ILPE (SEQ ID NO: 5),
a peptide consisting of amino acid sequence LPE,
a peptide consisting of amino acid sequence LP,
a peptide consisting of amino acid sequence LEP, and
a peptide consisting of amino acid sequence LE.
Item 5.

A cognitive function-improving composition comprising a peptide comprising amino acid sequence LP and/or a peptide comprising amino acid sequence LE.
Item 6.

The cognitive function-improving composition according to Item 5, wherein the number of amino acid residues that constitute the peptide comprising amino acid sequence LP and/or the peptide comprising amino acid sequence LE is 2 to 15.
Item 7.

The cognitive function-improving composition according to Item 5 or 6, wherein:

the peptide comprising amino acid sequence LP is at least one peptide selected from the group consisting of:
a peptide consisting of amino acid sequence ILPEY (SEQ ID NO: 1),
a peptide consisting of amino acid sequence ILELP (SEQ ID NO: 2),
a peptide consisting of amino acid sequence LPEY (SEQ ID NO: 4),
a peptide consisting of amino acid sequence ILPE (SEQ ID NO: 5),
a peptide consisting of amino acid sequence LPE, and
a peptide consisting of amino acid sequence LP; and

the peptide comprising amino acid sequence LE is at least one peptide selected from the group consisting of:
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LEP, and
a peptide consisting of amino acid sequence LE.
Item 8.

The cognitive function-improving composition according to any one of Items 1 to 7, which is an oral composition.
Item 9.

The cognitive function-improving composition according to any one of Items 1 to 8, which is a food and drink composition or a pharmaceutical composition.
Item 10.

A peptide consisting of amino acid sequence ILPEY (SEQ ID NO: 1), amino acid sequence ILELP (SEQ ID NO: 2), or amino acid sequence LYRGGLEP (SEQ ID NO: 3).
Item 11.

A cognitive function improvement method comprising administering a thermolysin digest of ovalbumin to a patient or potential group in need thereof.
Item 12.

The cognitive function improvement method according to Item 11, wherein the thermolysin digest of ovalbumin comprises a peptide comprising amino acid sequence LP and/or a peptide comprising amino acid sequence LE.
Item 13.

The method according to Item 12, wherein the number of amino acid residues that constitute the peptide comprising amino acid sequence LP and/or the peptide comprising amino acid sequence LE is 2 to 15.
Item 14.

The cognitive function improvement method according to Item 11, wherein the thermolysin digest of ovalbumin comprises at least one peptide selected from the group consisting of:
a peptide consisting of amino acid sequence ILPEY (SEQ ID NO: 1),
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LPEY (SEQ ID NO: 4),
a peptide consisting of amino acid sequence ILPE (SEQ ID NO: 5),
a peptide consisting of amino acid sequence LPE,
a peptide consisting of amino acid sequence LP,
a peptide consisting of amino acid sequence LEP, and
a peptide consisting of amino acid sequence LE.
Item 15.

A cognitive function improvement method comprising administering a composition comprising a peptide comprising amino acid sequence LP and/or a peptide comprising amino acid sequence LE to a patient or potential group in need thereof.
Item 16.

The cognitive function improvement method according to Item 15, wherein the number of amino acid residues that constitute the peptide comprising amino acid sequence LP and/or the peptide comprising amino acid sequence LE is 2 to 15.
Item 17.

The cognitive function improvement method according to Item 15 or 16, wherein:

the peptide comprising amino acid sequence LP is at least one peptide selected from the group consisting of:
a peptide consisting of amino acid sequence ILPEY (SEQ ID NO: 1),
a peptide consisting of amino acid sequence ILELP (SEQ ID NO: 2),
a peptide consisting of amino acid sequence LPEY (SEQ ID NO: 4),
a peptide consisting of amino acid sequence ILPE (SEQ ID NO: 5),
a peptide consisting of amino acid sequence LPE, and
a peptide consisting of amino acid sequence LP; and

the peptide comprising amino acid sequence LE is at least one peptide selected from the group consisting of:
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LEP, and
a peptide consisting of amino acid sequence LE.
Item 18.

Use of a thermolysin digest of ovalbumin for producing a medicine or food and drink for improving cognitive function.
Item 19.

The use according to Item 18, wherein the thermolysin digest of ovalbumin comprises a peptide comprising amino acid sequence LP and/or a peptide comprising amino acid sequence LE.
Item 20.

The use according to Item 19, wherein the number of amino acid residues that constitute the peptide comprising amino acid sequence LP and/or the peptide comprising amino acid sequence LE is 2 to 15.
Item 21.

The use according to Item 18, wherein the thermolysin digest of ovalbumin comprises at least one peptide selected from the group consisting of:
a peptide consisting of amino acid sequence ILPEY (SEQ ID NO: 1),
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LPEY (SEQ ID NO: 4),
a peptide consisting of amino acid sequence ILPE (SEQ ID NO: 5),
a peptide consisting of amino acid sequence LPE,
a peptide consisting of amino acid sequence LP,
a peptide consisting of amino acid sequence LEP, and
a peptide consisting of amino acid sequence LE.
Item 22.

Use of a peptide comprising amino acid sequence LP and/or a peptide comprising amino acid sequence LE for producing a medicine or food and drink for improving cognitive function.
Item 23.

The use according to Item 22, wherein the number of amino acid residues that constitute the peptide comprising amino acid sequence LP and/or the peptide comprising amino acid sequence LE is 2 to 15.
Item 24.

The use according to Item 22 or 23, wherein:

the peptide comprising amino acid sequence LP is at least one peptide selected from the group consisting of:
a peptide consisting of amino acid sequence ILPEY (SEQ ID NO: 1),
a peptide consisting of amino acid sequence ILELP (SEQ ID NO: 2),
a peptide consisting of amino acid sequence LPEY (SEQ ID NO: 4),
a peptide consisting of amino acid sequence ILPE (SEQ ID NO: 5),
a peptide consisting of amino acid sequence LPE, and
a peptide consisting of amino acid sequence LP; and

the peptide comprising amino acid sequence LE is at least one peptide selected from the group consisting of:
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LEP, and
a peptide consisting of amino acid sequence LE.

### Advantageous Effects of Invention

A cognitive function-improving composition is provided.

### BRIEF DESCRIPTION OF DRAWINGS

FIG. 1 shows the implementation details (habituation (Habituation), acquisition (Trial 1), and test (Trial 2)) of the novel object recognition test (ORT).

FIG. 2 shows the results of the above ORT. In the figure, “ND” means a normal diet group, “HFD” means a high-fat diet group, and “HFD+digest” means a group administered a high-fat diet and an ovalbumin enzymatic digest. The results are shown as the mean of the percentage (%) of the approach time to a novel object to the total approach time to two objects placed in a square open field. Error bars indicate standard error (n=5 to 10). * indicates that the P value for the high-fat diet group was less than 0.05 in Dunnett's multiple comparison test.

FIG. 3 shows the results of the above ORT. The results are shown as the mean (n=2 to 5) of the percentage (%) of each peptide administered orally, with the percentage of the approach time to a novel object in the high-fat diet group as 100%.

### DESCRIPTION OF EMBODIMENTS

Embodiments included in the present disclosure are each described in more detail below.

The composition included in the present disclosure preferably contains a thermolysin digest of ovalbumin as an active ingredient. In the present specification, the composition is also referred to as “the composition of the present disclosure.”

Ovalbumin (RefSeq ID NP_990483.2) is the main protein contained in egg white.

Thermolysin (EC3.4.24.27) is a neutral metal protease stable against heat, produced by the gram-positive bacterium Bacillus thermoproteolyticus.

The thermolysin digest of ovalbumin can be obtained by treating ovalbumin with thermolysin.

The temperature of treatment with thermolysin is not particularly limited as long as the enzymatic reaction with thermolysin proceeds. For example, the treatment temperature can be appropriately selected from 30 to 70° C., 30 to 60° C., 30 to 50° C., 30 to 40° C., 40 to 70° C., 50 to 65° C., and the like.

The treatment time varies depending on the treatment temperature and cannot be generalized, but can be appropriately selected from, for example, about 30 minutes to 48 hours, about 1 to 10 hours, and about 2 to 8 hours.

The pH during treatment is not particularly limited as long as the enzymatic reaction with thermolysin proceeds. The pH during treatment can be appropriately selected from, for example, a pH of about 6.5 to 8.5 and a pH of about 7 to 8.

The mass ratio of ovalbumin and thermolysin during treatment is not particularly limited as long as the enzymatic reaction with thermolysin proceeds. The mass ratio can be, for example, about 1000:1 to 5:1.

In addition to thermolysin, the thermolysin digest of ovalbumin may be treated with any enzymes within the range that does not impair the effect of the present disclosure. Any such enzymes can be used singly or in combination of two or more.

In the treatment with thermolysin, as needed, thermolysin may be inactivated by heating to a temperature at which thermolysin is inactivated (e.g., heating at a temperature exceeding 80° C. for about 5 to 60 minutes).

The thermolysin digest of ovalbumin may be purified, as needed. The purification method can be any of a wide range of methods known in this technical field.

The content of the thermolysin digest of ovalbumin in the composition of the present disclosure is not particularly limited, and can be appropriately set with a limit of 100 mass %. The thermolysin digest of ovalbumin preferably contains a peptide comprising amino acid sequence LP and/or a peptide comprising amino acid sequence LE.

The number of amino acid residues that constitute the peptide comprising amino acid sequence LP or the peptide comprising amino acid sequence LE may be, for example, 2 to 15, 2 to 13, 2 to 11, 2 to 9, 2 to 7, 2 to 6, or 2 to 5.

The thermolysin digest of ovalbumin preferably contains, for example, a peptide consisting of amino acid sequence ILPEY (SEQ ID NO: 1), a peptide consisting of amino acid sequence LEQ, a peptide consisting of amino acid sequence ILELP (SEQ ID NO: 2), a peptide consisting of amino acid sequence LYRGGLEP (SEQ ID NO: 3), a peptide consisting of amino acid sequence LPEY (SEQ ID NO: 4), a peptide consisting of amino acid sequence ILPE (SEQ ID NO: 5), a peptide consisting of amino acid sequence LPE, a peptide consisting of amino acid sequence LP, a peptide consisting of amino acid sequence LEP, a peptide consisting of amino acid sequence LE, and the like. These peptides may be contained singly or in combination of two or more.

As shown in the Examples provided later, the peptide consisting of amino acid sequence ILPEY (SEQ ID NO: 1), the peptide consisting of amino acid sequence LEQ, the peptide consisting of amino acid sequence ILELP (SEQ ID NO: 2), the peptide consisting of amino acid sequence LYRGGLEP (SEQ ID NO: 3), the peptide consisting of amino acid sequence LPEY (SEQ ID NO: 4), the peptide consisting of amino acid sequence ILPE (SEQ ID NO: 5), and the peptide consisting of amino acid sequence LEP are contained in the thermolysin digest of ovalbumin. That is, the thermolysin digest of ovalbumin more preferably contains the peptide consisting of amino acid sequence ILPEY (SEQ ID NO: 1), the peptide consisting of amino acid sequence LEQ, the peptide consisting of amino acid sequence ILELP (SEQ ID NO: 2), the peptide consisting of amino acid sequence LYRGGLEP (SEQ ID NO: 3), the peptide consisting of amino acid sequence LPEY (SEQ ID NO: 4), the peptide consisting of amino acid sequence ILPE (SEQ ID NO: 5), or the peptide consisting of amino acid sequence LEP.

The composition of the present disclosure also includes a composition comprising a peptide comprising amino acid sequence LP and/or a peptide comprising amino acid sequence LE. The peptide comprising amino acid sequence LP or the peptide comprising amino acid sequence LE can be used singly or in combination of two or more.

The number of amino acid residues that constitute the peptide comprising amino acid sequence LP or the peptide comprising amino acid sequence LE may be, for example, 2 to 15, 2 to 13, 2 to 11, 2 to 9, 2 to 7, 2 to 6, or 2 to 5.

In other words, an amino acid residue may be further added to amino acid sequence LP or amino acid sequence LE. The number of amino acid residues to be added may be, for example, 1 to 13, 1 to 11, 1 to 9, 1 to 7, 1 to 6, 1 to 5, 1 to 4, or 1 to 3.

The amino acid residue to be added is not particularly limited, and any amino acid residue can be added.

The amino acid residue to be added may be added to the N-terminal side or C-terminal side of amino acid sequence LP or amino acid sequence LE, or to both the N-terminal side and C-terminal side. The number of amino acid residues to be added to the N-terminal side may be, for example, 1 to 9, 1 to 7, 1 to 6, 1 to 5, 1 to 4, or 1 to 3. The number of amino acid residues to be added to the C-terminal side may be, for example, 1 to 4, 1 to 3, or 1 or 2.

Examples of the peptide comprising amino acid sequence LP include a peptide consisting of amino acid sequence ILPEY (SEQ ID NO: 1), a peptide consisting of amino acid sequence ILELP (SEQ ID NO: 2), a peptide consisting of amino acid sequence LPEY (SEQ ID NO: 4), a peptide consisting of amino acid sequence ILPE (SEQ ID NO: 5), a peptide consisting of amino acid sequence LPE, a peptide consisting of amino acid sequence LP, and the like.

Examples of the peptide comprising amino acid sequence LE include a peptide consisting of amino acid sequence LEQ, a peptide consisting of amino acid sequence ILELP (SEQ ID NO: 2), a peptide consisting of amino acid sequence LYRGGLEP (SEQ ID NO: 3), a peptide consisting of amino acid sequence LEP, a peptide consisting of amino acid sequence LE, and the like.

The peptide comprising amino acid sequence LP or the peptide comprising amino acid sequence LE may be, for example, a peptide consisting of an amino acid sequence having deletion, substitution, or addition of at least one amino acid residue in amino acid sequence ILPEY (SEQ ID NO: 1), amino acid sequence LEQ, amino acid sequence ILELP (SEQ ID NO: 2), amino acid sequence LYRGGLEP (SEQ ID NO: 3), amino acid sequence LPEY (SEQ ID NO: 4), amino acid sequence ILPE (SEQ ID NO: 5), amino acid sequence LPE, or amino acid sequence LEP.

In the amino acid sequence having deletion, substitution, or addition of at least one amino acid residue in amino acid sequence ILPEY (SEQ ID NO: 1), amino acid sequence LEQ, amino acid sequence ILELP (SEQ ID NO: 2), amino acid sequence LYRGGLEP (SEQ ID NO: 3), amino acid sequence LPEY (SEQ ID NO: 4), amino acid sequence ILPE (SEQ ID NO: 5), amino acid sequence LPE, or amino acid sequence LEP, the number of amino acid residues deleted, substituted, or added may be, for example, 1, 2, 3, 4, 5, 6, or 7. The number of amino acid residues deleted, substituted, or added may be, for example, 1 to 7, 1 to 6, 1 to 5, 1 to 4, 1 to 3, or 1 or 2.

When an amino acid residue is added, it may be added to the N-terminal side or C-terminal side.

When an amino acid residue is substituted, the substitution is preferably a conservative substitution. In the present specification, “conservative substitution” means substitution of an amino acid residue with another amino acid residue with a side chain having properties similar to those of the side chain of the amino acid residue. Specific examples of conservative substitutions include substitution between amino acid residues with basic side chains, such as lysine, arginine, and histidine; substitution between amino acid residues with acidic side chains, such as aspartic acid and glutamic acid; substitution between amino acid residues with uncharged polar side chains, such as glycine, asparagine, glutamine, serine, threonine, tyrosine, and cysteine; substitution between amino acid residues with non-polar side chains, such as alanine, valine, leucine, isoleucine, proline, phenylalanine, methionine, and tryptophan; substitution between amino acid residues with β-branched side chains, such as threonine, valine, and isoleucine; substitution between amino acid residues with aromatic side chains, such as tyrosine, phenylalanine, tryptophan, and histidine; and the like.

The technique of adding mutations, such as deletion, substitution, or addition of at least one amino acid residue, to a specific amino acid sequence is well known in this technical field and can be performed using any method. When adding mutations, such as deletion, substitution, or addition of at least one amino acid residue, to a specific amino acid sequence, for example, restriction enzyme treatment, treatment with exonuclease or DNA ligase, site-specific mutagenesis, or random mutagenesis can be used.

The peptide comprising amino acid sequence LP or the peptide comprising amino acid sequence LE may be composed only of L-type amino acid residues, D-type amino acid residues, or a mixture of both. Further, the peptide comprising amino acid sequence LP or the peptide comprising amino acid sequence LE may be composed only of naturally occurring amino acid residues, or may be composed only of modified amino acid residues with an arbitrary functional group attached, such as phosphorylated or glycosylated amino acids, or may be a mixture of both. When the peptide contains two or more asymmetric carbon atoms, the peptide may be an enantiomer, a diastereomer, or a mixture of both.

The peptide comprising amino acid sequence LP or the peptide comprising amino acid sequence LE may be in the form of a salt (an acid addition salt or a basic salt). Examples of acid addition salts include inorganic acid salts, such as hydrochloride, sulfate, nitrate, phosphate, hydrobromide, and perchlorate; salts of organic acids, such as citric acid, succinic acid, maleic acid, fumaric acid, malic acid, tartaric acid, p-toluenesulfonic acid, benzenesulfonic acid, methanesulfonic acid, and trifluoroacetic acid; and the like. Examples of basic salts include salts of alkali metals, such as sodium, potassium, and lithium; salts of alkaline earth metals, such as calcium and magnesium; and the like. Examples of bases that can be used to produce base addition salts include sodium hydroxide, potassium hydroxide, lithium hydroxide, calcium hydroxide, and the like. Acids and bases capable of producing pharmaceutically acceptable salts are described in Stahland Wermuth, eds., 2008, Handbook of Pharmaceutical Salts: Properties, Selection and Use, Verlag Helvetica Chimica Acta, Zurich, Switzerland.

The peptide comprising amino acid sequence LP or the peptide comprising amino acid sequence LE may be in the form of a solvate. Examples of solvates include water (for hydrates) and solvates with methanol, ethanol, isopropanol, acetic acid, tetrahydrofuran, acetone, dimethylformamide, dimethylsulfoxide, dimethylacetamide, acetamide, ethylene glycol, propylene glycol, dimethoxyethane, etc.

The peptide comprising amino acid sequence LP or the peptide comprising amino acid sequence LE may be synthesized by any known peptide synthesis method, enzyme method, or the like. Further, the peptide comprising amino acid sequence LP or the peptide comprising amino acid sequence LE may be obtained from a microorganism or cultured cell that has been engineered to produce the peptide comprising amino acid sequence LP or the peptide comprising amino acid sequence LE, for example, from a microorganism or cultured cell that has been inserted with a gene encoding the peptide, or may be obtained by in vitro translation.

The peptide comprising amino acid sequence LP or the peptide comprising amino acid sequence LE may be one obtained by chemically or enzymatically hydrolyzing a protein or polypeptide raw material derived from egg, milk, soybeans, wheat, egg, meat, fish meat, seafood, etc. As shown in the Examples provided later, the peptide consisting of amino acid sequence ILPEY (SEQ ID NO: 1), the peptide consisting of amino acid sequence LEQ, the peptide consisting of amino acid sequence ILELP (SEQ ID NO: 2), the peptide consisting of amino acid sequence LYRGGLEP (SEQ ID NO: 3), the peptide consisting of amino acid sequence LPEY (SEQ ID NO: 4), the peptide consisting of amino acid sequence ILPE (SEQ ID NO: 5), and the peptide consisting of amino acid sequence LEP are contained in the thermolysin digest of ovalbumin. Therefore, the peptide comprising amino acid sequence LP or the peptide comprising amino acid sequence LE can be obtained, for example, by digesting ovalbumin with thermolysin.

The content of the peptide comprising amino acid sequence LP or the peptide comprising amino acid sequence LE in the composition of the present disclosure is not particularly limited, and can be appropriately set with a limit of 100 mass %.

The composition of the present disclosure contains at least one selected from the group consisting of a thermolysin digest of ovalbumin, a peptide comprising amino acid sequence LP, and a peptide comprising amino acid sequence LE, and may further contain other components. Examples of other such components include pharmaceutically or food hygienically acceptable bases, carriers, solvents, dispersants, emulsifiers, buffers, stabilizers, excipients, binders, disintegrants, lubricants, thickeners, antioxidants, preservatives, coating agents, coloring agents, gastric mucosa protective agents, and other components, as well as components and materials that can be used as foods etc. These components can be used singly or in combination of two or more.

The form of the composition of the present disclosure is not particularly limited, and examples include tablets, pills, capsules, powders, fine granules, granules, liquids, troches, jellies, injections, plasters, extracts, suppositories, suspensions, tinctures, ointments, poultices, nasal drops, inhalants, liniments, lotions, aerosols, and the like.

The composition of the present disclosure can be prepared by combining at least one selected from the group consisting of a thermolysin digest of ovalbumin, a peptide comprising amino acid sequence LP, and a peptide comprising amino acid sequence LE, and optionally other components, according to a conventional method.

The composition of the present disclosure is preferably an oral composition. Specific examples include food and drink compositions, pharmaceutical compositions, and the like. Food and drink compositions may be, for example, functional food, dietary supplements, supplements, food for health use, food for specified health use, food with nutrient function claims, and food with functional claims.

As shown in the Examples provided later, the composition of the present disclosure has a cognitive function improvement effect, for example. Therefore, the composition of the present disclosure can be suitably used, for example, as a cognitive function-improving composition.

In the present specification, “cognitive function” means cognitive function related to memory evaluated by the novel object recognition test (ORT), which is at least known as a behavioral test to evaluate cognitive function in mammals. Further, “cognitive function improvement” means suppressing and improving the decline in cognitive function, particularly brain function related to memory. In the present specification, the term “suppressing” includes the meaning of prevention or avoidance. Further, the term “improvement” includes the meaning of maintenance or enhancement. That is, the term “cognitive function improvement” in the present specification includes at least one of the following: suppressing the decline in cognitive function, suppressing the decline in cognitive function to maintain the cognitive function, and improving cognitive function so that the cognitive function does not decline (or to maintain the cognitive function). The term “cognitive function improvement” also includes, for example, recovery of cognitive abilities that have declined or symptoms that show signs of decline.

Examples of the method of administering the composition of the present disclosure include oral administration and parenteral administration. Examples of parenteral administration include intravenous, intraarterial, intramuscular, subcutaneous, intraperitoneal, rectal, transdermal, and local administration. Preferred among these is oral administration.

Subjects to which the composition of the present disclosure is administered, or given for ingestion, are not particularly limited, and examples include humans and non-human mammals. Examples of non-human mammals include rats, mice, rabbits, cows, pigs, dogs, cats, sheep, monkeys, and the like.

Examples of target humans include dementia patients or people suspected of having dementia, people with mild cognitive impairment (MCI) or people suspected of having mild cognitive impairment, elderly people (people aged 65 or older), people with two or more of these characteristics, and the like. These people correspond to patients or potential patients in need of the composition of the present disclosure.

The dosage or intake of the composition of the present disclosure is not particularly limited, and is determined according to the age, weight, and sex of the subject, the administration method, the type of active ingredient, and the like.

For example, when the active ingredient is a thermolysin digest of ovalbumin, the dosage of the active ingredient is, for example, 1 mg/kg to 50000 mg/kg, preferably 5 mg/kg to 10000 mg/kg, and more preferably 10 to 3000 mg/kg, per day for an adult.

For example, when the active ingredient is a peptide comprising amino acid sequence LP and/or a peptide comprising amino acid sequence LE, the dosage of the active ingredient is, for example, 0.01 mg/kg to 500 mg/kg, preferably 0.05 mg/kg to 100 mg/kg, and more preferably, 0.1 to 30 mg/kg, per day for an adult.

The composition of the present disclosure may be administered or taken once a day, or may be administered or taken multiple times a day, for example, 2 to 5 times.

The present disclosure also includes a peptide consisting of amino acid sequence ILPEY (SEQ ID NO: 1).

Further, the present disclosure also includes a peptide consisting of amino acid sequence ILELP (SEQ ID NO: 2).

Further, the present disclosure also includes a peptide consisting of amino acid sequence LYRGGLEP (SEQ ID NO: 3).

For these peptides, the above description can be incorporated.

In the present specification, the term “comprising” includes “consisting essentially of” and “consisting of.” Further, the present disclosure includes all of any combinations of the constituent requirements described in the present specification.

In addition, the various numerical values or characteristics described in each embodiment of the present disclosure described above may be combined in any way in specifying the subject matters included in the present invention. In other words, the present disclosure includes all the subject matters comprising all combinations of the combinable characteristics described in the present specification.

### EXAMPLES

The contents of the present disclosure are described in detail below using the following Examples. However, the present disclosure is not limited thereto. In the following, tests are conducted under atmospheric pressure and room temperature conditions, unless otherwise specified.

It is known that feeding mice a high-fat diet for one week results in a decline in cognitive function (NPL 1). Based on this knowledge, the present inventors examined whether cognitive function decline induced by short-term high-fat diet intake could be improved by an enzymatic digest of ovalbumin.

### Production of Enzymatic Digest of Ovalbumin

A 20 mg/mL ovalbumin (Albumin from chicken egg white lyophilized powder (produced by Sigma-Aldrich)) aqueous solution was boiled, and then thermolysin (produced by Nacalai Tesque, Inc.) was added so that E/S (enzyme/substrate)=1/100, followed by digestion at 37° C. for 5 hours. During the reaction, the pH was adjusted to 7.5. After completion of the reaction, the enzymatic reaction was stopped by boiling again, followed by centrifugation, and the supernatant was lyophilized.

### Test Animals

11-week-old male ddY mice (Japan SLC) were pre-fed for one week in a feedlot set up on a 12-hour light/dark cycle. Next, the mice were fed a fat 60% kcal diet (D12492, Research Diets) as a high-fat diet (HFD) for one week, and then a behavioral test was conducted. The ovalbumin enzymatic digest (30 mg/kg/day) was orally administered for three days from the fifth to seventh day in the one week of feeding the mice a high-fat diet.

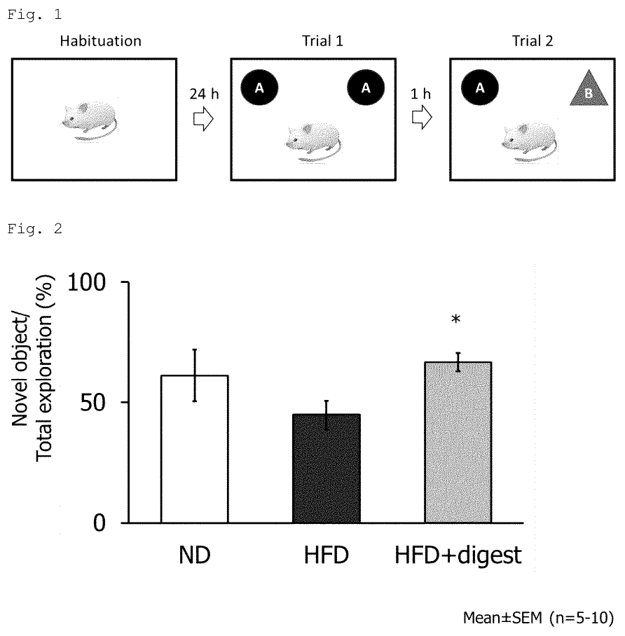

### Novel Object Recognition Test (ORT, FIG. 1)

Cognitive function was evaluated in the ORT (NPL 1, Leger et al., Nat Protoc. December 2013; 8(12): 2531-7). The ORT includes 3 steps (habituation (Habituation), acquisition (Trial 1), and test (Trial 2)). The habituation step was performed on the sixth day by leaving the mice in a square open field for 5 minutes. Next, 24 hours after the habituation step, the acquisition step was performed. The acquisition step was performed by placing the mice in an open field in which two identical objects were placed. Further, 1 hour after the acquisition step, the test step was performed. In the test step, one of the two identical objects was replaced with a novel object. The percentage of the exploration time to the novel object in the test step was evaluated based on the mice's approach time to each object. It was determined that cognitive function increased when the percentage of the exploration time to the novel object increased. The results are shown in FIG. 2.

As shown in FIG. 2, it was revealed that when the ovalbumin enzymatic digest (30 mg/kg/day) was orally administered, the percentage of the approach time to the novel object significantly increased in the ORT. Therefore, the ovalbumin enzymatic digest was found to improve the cognitive function that had declined due to the high-fat diet.

The ORT was performed in the same manner as described above, except that the following peptides (3 mg/kg/day (2 mg/kg/day only for LEQ)) were used in place of the ovalbumin enzymatic digest.

The peptides used were a peptide consisting of amino acid sequence ILPEY (SEQ ID NO: 1), a peptide consisting of amino acid sequence LEQ, a peptide consisting of amino acid sequence ILELP (SEQ ID NO: 2), a peptide consisting of amino acid sequence LYRGGLEP (SEQ ID NO: 3), a peptide consisting of amino acid sequence LPEY (SEQ ID NO: 4), a peptide consisting of amino acid sequence ILPE (SEQ ID NO: 5), a peptide consisting of amino acid sequence LPE, a peptide consisting of amino acid sequence LP, a peptide consisting of amino acid sequence LEP, and a peptide consisting of amino acid sequence LE.

It was confirmed that the peptide consisting of amino acid sequence ILPEY (SEQ ID NO: 1), the peptide consisting of amino acid sequence LEQ, the peptide consisting of amino acid sequence ILELP (SEQ ID NO: 2), the peptide consisting of amino acid sequence LYRGGLEP (SEQ ID NO: 3), the peptide consisting of amino acid sequence LPEY (SEQ ID NO: 4), the peptide consisting of amino acid sequence ILPE (SEQ ID NO: 5), and the peptide consisting of amino acid sequence LEP were contained in the ovalbumin enzymatic digest described above.

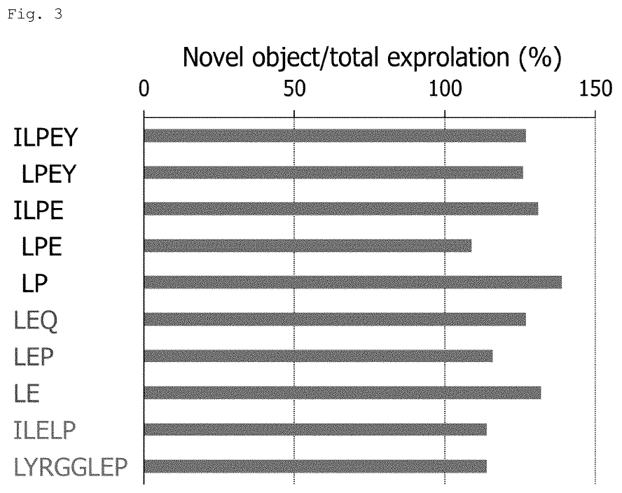

FIG. 3 shows the percentage (%) of the approach time to the novel object in the oral administration of each peptide with respect to the approach time to the novel object in the high-fat diet group.

As shown FIG. 3, it was revealed that when any peptide was used, the percentage of the approach time to the novel object increased in the ORT. Therefore, the peptides described above were found to improve the cognitive function that had declined due to the high-fat diet.

### Sequence Listing

1-9. (canceled)

10. A peptide consisting of amino acid sequence ILPEY (SEQ ID NO: 1), amino acid sequence ILELP (SEQ ID NO: 2), or amino acid sequence LYRGGLEP (SEQ ID NO: 3).

11. A method for improving cognitive function in a subject in need thereof comprising:
administering a composition comprising a thermolysin digest of ovalbumin to the subject.

12. The method according to claim 11, wherein the thermolysin digest of ovalbumin comprises a peptide comprising amino acid sequence LP and/or a peptide comprising amino acid sequence LE.

13. The method according to claim 12, wherein a number of amino acid residues that constitute the peptide comprising amino acid sequence LP and/or the peptide comprising amino acid sequence LE is 2 to 15.

14. The method according to claim 11, wherein the thermolysin digest of ovalbumin comprises at least one peptide selected from the group consisting of:
a peptide consisting of amino acid sequence ILPEY (SEQ ID NO: 1),
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LPEY (SEQ ID NO: 4),
a peptide consisting of amino acid sequence ILPE (SEQ ID NO: 5),
a peptide consisting of amino acid sequence LPE,
a peptide consisting of amino acid sequence LP,
a peptide consisting of amino acid sequence LEP, and
a peptide consisting of amino acid sequence LE.

15. A method for improving cognitive function in a subject in need thereof comprising:
administering a peptide comprising amino acid sequence LP and/or a peptide comprising amino acid sequence LE to the subject.

16. The method according to claim 15, wherein a number of amino acid residues that constitute the peptide comprising amino acid sequence LP and/or the peptide comprising amino acid sequence LE is 2 to 15.

17. The method according to claim 15, wherein:
the peptide comprising amino acid sequence LP is at least one peptide selected from the group consisting of:
a peptide consisting of amino acid sequence ILPEY (SEQ ID NO: 1),
a peptide consisting of amino acid sequence ILELP (SEQ ID NO: 2),
a peptide consisting of amino acid sequence LPEY (SEQ ID NO: 4),
a peptide consisting of amino acid sequence ILPE (SEQ ID NO: 5),
a peptide consisting of amino acid sequence LPE, and
a peptide consisting of amino acid sequence LP; and
the peptide comprising amino acid sequence LE is at least one peptide selected from the group consisting of:
a peptide consisting of amino acid sequence LEQ,
a peptide consisting of amino acid sequence ILELP (SEQ ID NO: 2),
a peptide consisting of amino acid sequence LYRGGLEP (SEQ ID NO: 3),
a peptide consisting of amino acid sequence LEP, and
a peptide consisting of amino acid sequence LE.

18. The method according to claim 11, wherein the composition is administered orally.

19. The method according to claim 11, wherein the composition is administered in a form of a food and drink composition or a pharmaceutical composition.
